(19) United States
(12) Patent Application Publication
(10) Pub. No.: US 2025/0082866 A1
(43) Pub. Date: Mar. 13, 2025

(54) AEROSOL MEDICAMENT DELIVERY SYSTEM AND CONTROL METHOD THEREFOR

(71) Applicant: DELBio, INC., TAOYUAN CITY (TW)

(72) Inventors: I CHEN MA, TAOYUAN CITY (TW); SHENG WEN PAI, TAOYUAN CITY (TW); CHI CHANG NIEN, TAOYUAN CITY (TW)

(73) Assignee: DELBio, INC., TAOYUAN CITY (TW)

(21) Appl. No.: 18/826,209

(22) Filed: Sep. 6, 2024

Related U.S. Application Data
(60) Provisional application No. 63/536,944, filed on Sep. 7, 2023.

(51) Int. Cl. A61M 11/00

(52) U.S. Cl. CPC A61M 11/001; A61M 2205/3331; A61M 2205/3386; A61M 2205/70; A61M 2230/42

(57) ABSTRACT

An aerosol medicament delivery system is provided, which includes a first pressure sensor configured to detect external air pressure outside the aerosol medicament delivery system; a second pressure sensor configured to detect air pressure in an airway of the aerosol medicament delivery system; a controller; a driver module, a nebulization module including a mesh, wherein the controller controls the driver module to drive the nebulization module for performing nebulization based on air pressure difference between the external air pressure and the air pressure in the airway; a detection module configured to detect pressure applied by a medicament onto the mesh, convert the pressure into a voltage signal or a current signal, and transmit the voltage signal or the current signal to the controller; and a power supply module configured to provide power for the aerosol medicament delivery system.

### FIELD OF INVENTION

The present disclosure relates to an aerosol medicament delivery system and control methods therefor.

### BACKGROUND OF INVENTION

In recent years, aerosol medicament delivery systems have been widely used in medical care. Nebulizers are a type of aerosol medicament delivery system that can nebulize inhalation liquid-type liquid medicaments into gas to improve the convenience of inhalation treatment for patients.

The flow sensing technology of dual pressure sensors is used in conventional nebulizers. The two pressure sensors are arranged inside the flow channel of the nebulizer to achieve differential pressure calculation, and the output signal of the nebulizer is a flow signal, but the disadvantage is that it is more expensive than a nebulizer using a single pressure sensor. In addition, there are also nebulizers using a single pressure sensor. However, when the sensing value of the single pressure sensor deviates in the environment, or the pressure value cannot regress normally, inaccuracy and failure may be caused, resulting in a waste of a medicament and a low dosage rate. Furthermore, the nebulizers with conventional single pressure sensor and flow sensor are expensive and large in size, requiring more space to be placed in the nebulizer.

Therefore, how to provide a nebulizer that is low in cost, easy to carry, does not cause a waste of the medicament and can increase the dosage rate has become an urgent problem that needs to be solved.

### SUMMARY OF INVENTION

In view of the above, the primary object of the present disclosure is to provide an aerosol medicament delivery system, including:
a first pressure sensor configured to detect external air pressure outside the aerosol medicament delivery system;
a second pressure sensor configured to detect air pressure in an airway of the aerosol medicament delivery system;
a controller;
a driver module;
a nebulization module including a mesh, wherein the controller controls the driver module to drive the nebulization module for performing nebulization based on air pressure difference between the external air pressure and the air pressure in the airway;
a detection module configured to detect pressure applied by a medicament onto the mesh, convert the pressure into a voltage signal or a current signal, and transmit the voltage signal or the current signal to the controller, wherein the controller determines the liquid level of the medicament based on the voltage signal or the current signal; and
a power supply module configured to provide power for the aerosol medicament delivery system.

In some embodiments, under a condition that the air pressure difference between the external air pressure and the air pressure in the airway is greater than a first threshold, the controller drives the nebulization module to perform nebulization.

In some embodiments, the first threshold is 60 Pa.

In some embodiments, the voltage signal or the current signal is detected and fed back from the nebulization module when the nebulization module is being operated; and whether the medicament is exhausted based on a change of the voltage signal or the current signal is determined.

In some embodiments, when it is determined that the medicament is exhausted, the nebulization is stopped; and when it is determined that the medicament is not yet exhausted, the nebulization is continuing.

In some embodiments, it is determined that the medicament is exhausted when a slope of the voltage signal or the current signal corresponding to time is greater than a second threshold.

In some embodiments, the second threshold is 1.

Another object of the present disclosure is to provide a method for offloading an aerosol medicament delivery system, wherein the aerosol medicament delivery system includes a controller and a driver module configured to drive a nebulization module for operation, and the method includes steps of:
S201: activating the aerosol medicament delivery system;
S202: nebulizing a medicament by using the nebulization module; and
S203: adjusting the driver module by the controller to reduce an operating voltage or an operating current of the nebulization module, or increase or reduce an operating frequency of the nebulization module after the nebulization module is operated for a predetermined time.

In some embodiments, after S201 and before S202, the method further includes a step of:
S201A: detecting a first operating frequency, a second operating frequency and a third operating frequency of the driver module by the controller after activating the aerosol medicament delivery system;
wherein S202 further includes controlling the driver module to drive the nebulization module by the controller to nebulize the medicament at the first operating frequency;
wherein S203 further includes controlling the driver module to drive the nebulization module by the controller to nebulize the medicament at the second operating frequency after the nebulization module is operated for the predetermined time, thereby avoiding overheating of the medicament and causing failure of the medicament.

In some embodiments, a current value corresponding to the first operating frequency is highest, a current value corresponding to the second operating frequency is second highest, and a current value corresponding to the third operating frequency is lower than the current value corresponding to the second operating frequency.

In some embodiments, the third operating frequency is not used for nebulization.

In some embodiments, the aerosol medicament delivery further includes a detection module, and the method further includes steps of:
S303: providing a voltage signal or a current signal fed back from the nebulization module for the detection module when the nebulization module is operated;
S304: determining a liquid level of the medicament by the controller based on a change of the voltage signal or the current signal; and
S305: adjusting the driver module by the controller to reduce a voltage, reduce a current, or increase or reduce a frequency when the liquid level of the medicament is lower than a predetermined value, thereby avoiding overheating of the medicament and causing failure of the medicament.

In some embodiment, wherein S305 further includes: controlling the driver module to drive the nebulization module by the controller to nebulize the medicament at the second operating frequency when the liquid level of the medicament is lower than the predetermined value, thereby avoiding overheating of the medicament and causing failure of the medicament.

In some embodiments, a current value corresponding to the first operating frequency is highest, a current value corresponding to the second operating frequency is second highest, and a current value corresponding to the third operating frequency is lower than the current value corresponding to the second operating frequency.

In some embodiments, the third operating frequency is not used for nebulization.

Yet another object of the present disclosure is to provide a method for controlling nebulization of an aerosol medicament delivery system, and the method includes steps of:
S401: detecting a respiratory traction pressure of the aerosol medicament delivery system through a first pressure sensor and a second pressure sensor by a controller,
   detecting several inhalation cycles and exhalation cycles when the respiratory traction pressure exceeds a threshold to calculate an average time of the inhalation cycles and an average time of the exhalation cycles, and multiply the average time of the inhalation cycles and the average time of the exhalation cycles by a set ratio respectively as an adjusted average time of the inhalation cycles and an adjusted average time of the exhalation cycles;
S402: determining whether a user is currently in the inhalation cycles or the exhalation cycles based on a pressure difference detected by the first pressure sensor and the second pressure sensor;
S403: during the inhalation cycles, stopping nebulization to reduce waste of medicament when inhalation time of the user exceeds the adjusted average time of the inhalation cycles, and
   continuing nebulization when the inhalation time of the user does not exceed the adjusted average time of the inhalation cycles;

S404: during the exhalation cycle, performing nebulization in advance to compensate insufficiency of the medicament caused by a delay when exhalation time of the user exceeds the adjusted average time of the exhalation cycles, and

stopping nebulization when the exhalation time of the user does not exceed the adjusted average time of the exhalation cycles; and
S405: updating the average time of the inhalation cycles, the average time of the exhalation cycles, the adjusted average time of the inhalation cycles and the adjusted average time of the exhalation cycles after the inhalation cycles and the exhalation cycles end.

In some embodiments, before S401, the method further includes steps of:
S401A: activating the aerosol medicament delivery system, controlling a driver module by a controller to drive a mesh at several different operating frequencies, and
   detecting voltage signals and current signals fed back from the mesh corresponding to the several different operating frequencies through a detection module;
S401B: determining the mesh of the nebulization module is operated normally when several different voltage signals or current signals fed back from the mesh corresponding to the several different operating frequencies are detected by the detection module; and
S401C: determining the mesh of the nebulization module is operated abnormally when consistent voltage signals or current signals fed back from the mesh corresponding to the several different operating frequencies are detected by the detection module.

In some embodiments, the set ratio is 80% of the average time of the inhalation cycles.

In some embodiments, before S401, the method further includes a step of:
S401′: performing calibration of the first pressure sensor and the second pressure sensor by the controller before the user inhales and exhales.

In some embodiments, the voltage signals and current signals fed back from the mesh corresponding to the several different operating frequencies are not affected by the dosage of the medicament.

Compared with prior art, the advantages of the aerosol medicament delivery system of the present disclosure are as follows:
(1) small size and low costs;
(2) the voltage or current can be adjusted automatically after a specific period of time, or the voltage or current can be adjusted through a change of the liquid level of the medicament fed back from the mesh to reduce the waste caused by overheating of the medicament;
(3) dual pressure sensors are used to calibrate each other to prevent one of the pressure sensors from being out of accuracy and unable to regress normally, causing abnormal nebulization; and
(4) the time to start nebulization in advance and stop nebulization in advance can be adjusted dynamically based on the user's inhalation cycles and exhalation cycles to avoid unnecessary loss of the medicament.

### DESCRIPTION OF DRAWINGS

For a more complete understanding of the embodiments and

their advantages, reference is now made to the following description taken in conjunction with the accompanying drawings, in which:

FIG. 1 shows the composition of an aerosol medicament delivery system 10 of the present disclosure;

FIG. 2 shows the relationship between a voltage signal and a current signal fed back from the medicament and nebulization module 105;

FIG. 3 is a flow chart for offloading aerosol medicament delivery system 10 according to an embodiment of the present disclosure;

FIGS. 4A and 4B respectively show driving levels of aerosol medicament delivery system 10 of the present disclosure after a voltage or a current of driver module 104 is reduced after a specific time, and the nebulization effect of aerosol medicament delivery system 10 of the present disclosure when an operating frequency of driver module 104 is adjusted after a specific time;

FIG. 5 is a flow chart for offloading aerosol drug delivery system 10 according to another embodiment of the present disclosure;

FIGS. 6A and 6B are respectively a schematic diagram of various liquid levels of the medicament in nebulization module 105, and the feedback voltage signals and feedback current signals of various liquid levels of the medicament in nebulization module 105 on a reaction force of the mesh;

FIG. 7 is a flow chart for controlling nebulization of aerosol medicament delivery system 10 of the present disclosure;

FIG. 8 is a graph showing that nebulization module 105 starts nebulization in advance and stops nebulization in advance through a pressure difference between a measurement value of the first pressure sensor and a measurement value of the second pressure sensor;

FIGS. 9A and 9B respectively show the pressure calibration situation of a conventional nebulizer using a single pressure sensor and the pressure calibration situation of aerosol medicament delivery system 10 of the present disclosure using dual pressure sensors; and

FIGS. 10A and 10B respectively show that the nebulization module in normal operation reflects different current signals at different operating frequencies, and the nebulization module in abnormal operation reflects consistent current signals at different operating frequencies.

### DETAILED DESCRIPTION OF PREFERRED EMBODIMENTS

Some specific embodiments according to the present disclosure will be described below; however, without departing from the spirit of the present disclosure, the present disclosure can still be practiced in various forms, and the protection scope of the present disclosure should not be construed as being limited to what is stated in the specification. In addition, unless otherwise indicated in the context, “a”, “the” and similar terms used in the specification (especially in the appended claims) should be understood to include both the singular and the plural forms.

Although a series of operations or steps are used below to illustrate the method disclosed herein, the order shown in these operations or steps should not be construed as a limitation of the present disclosure. For example, certain operations or steps may be performed in a different order and/or concurrently with other steps. Furthermore, not all operations, steps, and/or features must be performed to practice embodiments of the present disclosure. Additionally, each operation or step described herein may include several sub-steps or actions.

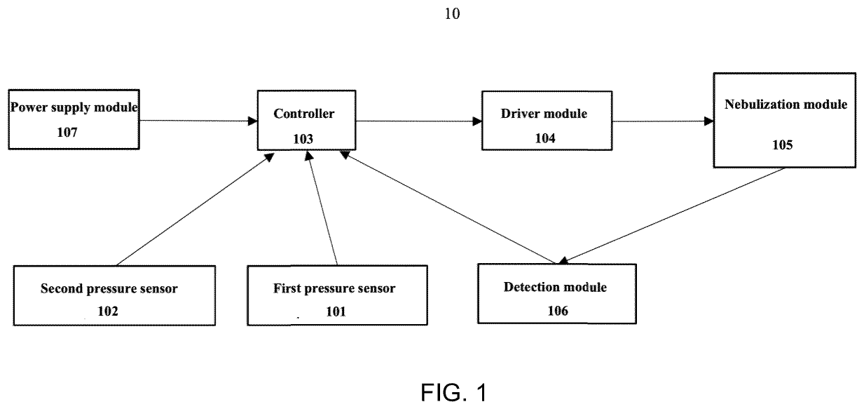

Referring to FIG. 1 first, one embodiment of the present disclosure provides an aerosol medicament delivery system 10, including a first pressure sensor 101, a second pressure sensor 102, a controller 103, a driver module 104, a nebulization module 105, a detection module 106 and a power supply module 107. First pressure sensor 101 is configured to detect external air pressure outside the aerosol medicament delivery system 10. Second pressure sensor 102 is configured to detect air pressure in an airway of aerosol medicament delivery system 10. Nebulization module 105 includes a mesh, and controller 103 controls driver module 104 to drive nebulization module 105 for performing nebulization based on air pressure difference between the external air pressure and the air pressure in the airway. Detection module 106 is configured to detect pressure applied by a medicament onto the mesh, convert the pressure into a voltage signal or a current signal, and transmit the voltage signal or the current signal to controller 103. Controller 103 determines the liquid level of the medicament based on the voltage signal or the current signal. Power supply module 107 is configured to provide power for aerosol medicament delivery system 10.

Specifically, nebulization module 105 connects to the positive and negative electrodes, and detection module 106 electrically connects to nebulization module 105 through the positive and negative electrodes. When nebulization module 105 is activated, the nebulized medicament causes a reaction force, and detection module 106 may capture the feedback voltage signal or current signal of nebulization module 105 through the positive and negative electrodes. When the medicament is almost to be exhausted, the force may reduce, causing the feedback voltage signal or current signal of nebulization module 105 to increase.

In a specific embodiment, when the air pressure difference between the external air pressure and the air pressure in the airway is greater than a first threshold (e.g., 60 Pa), controller 103 may drive the nebulization module 105 to perform nebulization.

In a specific embodiment, the voltage signal or the current signal is detected and fed back from the nebulization module when the nebulization module is being operated; and whether the medicament is exhausted based on a change of the voltage signal or the current signal is determined.

In a specific embodiment, when it is determined that the medicament is exhausted, the nebulization is stopped; and when it is determined that the medicament is not yet exhausted, the nebulization is continuing.

In a specific embodiment, it is determined that the medicament is exhausted when a slope of the voltage signal or the current signal corresponding to time is greater than a second threshold (e.g., 1).

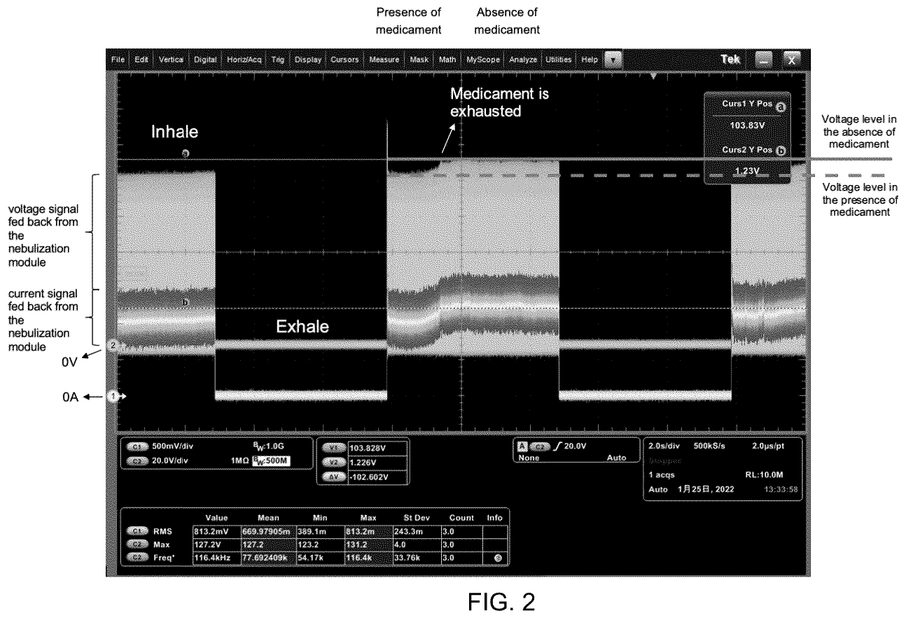

Specifically, FIG. 2 shows the relationship between a voltage signal and a current signal fed back from the medicament and nebulization module 105, and nebulization module 105 is operated at a fixed operating frequency. As shown in FIG. 2, when nebulization module 105 is operated, detection module 106 continuously obtains the feedback voltage signal and current signal from nebulization module 105. If there is an offset in the voltage signal and/or current signal, controller 103 captures the feedback voltage signal and/or the current signal to perform a slope calculation. When the slope of the voltage or current versus time is higher than 1, controller 103 determines that the medicament is almost to be exhausted, and further instructs nebulization module 105 to stop being operated or shut down. When the slope is lower than 1, controller 103 determines that the medicament is not yet exhausted and instructs nebulization module 105 to continue operation. The voltage signal and current signal fed back from nebulization module 105 may not cause an erroneous determination of aerosol medicament delivery system 10 due to the pause in exhalation, and the state can be continued. Therefore, the present disclosure can confirm whether the medicament is present through the difference between the feedback voltage signal and current signal, and can detect and control nebulization module 105 of aerosol medicament delivery system 10 through the feedback voltage signal and current signal.

The present disclosure provides various control methods for aerosol medicament delivery system 10, including offloading mechanisms of aerosol medicament delivery system 10, which will be described below with specific examples.

### EXAMPLE 1

### Offloading with Time

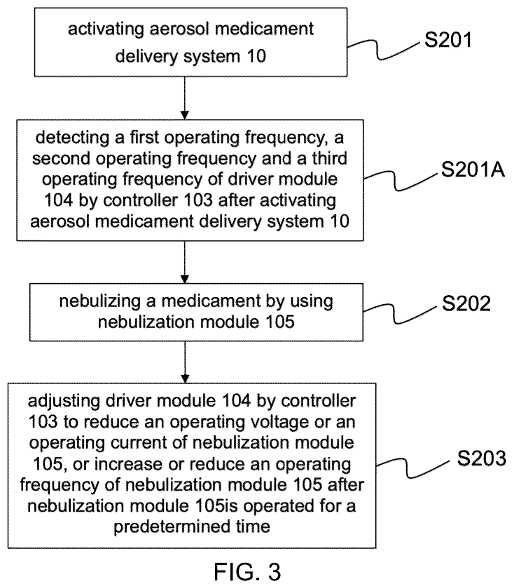

It is known that the nebulizer is driven by a constant power when operating. When the medicament is nebulized for a period of time, the power driven to the nebulization module does not decrease as the amount of the medicament decreases. Thus, the heat energy of the nebulization module may cause overheating and failure of the remaining medicament. In order to reduce the waste caused by overheating of the medicament, as shown in FIG. 3, the present disclosure provides a method for offloading aerosol medicament delivery system 10 including controller 103 and driver module 104 configured to drive nebulization module 105 for operation, and the method includes steps of:
S201: activating aerosol medicament delivery system 10;
S202: nebulizing the medicament by using nebulization module 105; and
S203: adjusting driver module 104 by controller 103 to reduce an operating voltage or an operating current of nebulization module 105, or increase or reduce an operating frequency of nebulization module 105 after nebulization module 105 is operated for a predetermined time.

In a specific embodiment, after S201 and before S202, the method may further include S201A: detecting a first operating frequency, a second operating frequency and a third operating frequency of driver module 104 by controller 103 after activating aerosol medicament delivery system 10. Preferably, S202 may further include controlling driver module 104 to drive nebulization module 105 by controller 103 to nebulize the medicament at the first operating frequency. Preferably, S203 may further include controlling driver module 104 to drive nebulization module 105 by controller 103 to nebulize the medicament at the second operating frequency after nebulization module 105 is operated for the predetermined time, thereby avoiding overheating of the medicament and causing failure of the medicament.

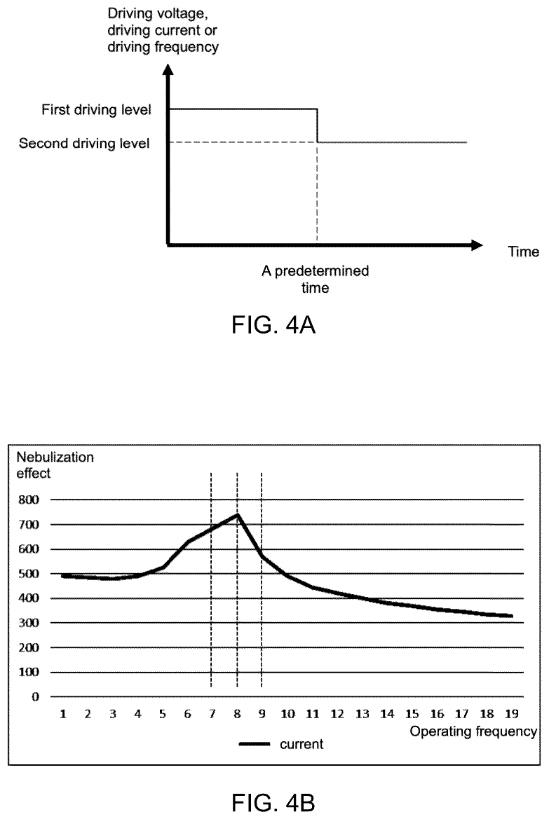

Specifically, the offloading mechanism includes that aerosol medicament delivery system 10 nebulizes all the medicament when the amount of the medicament is 0.1 ml to 0.3 ml; and since there is a built-in timer in aerosol medicament delivery system 10, the voltage or current of driver module 104 is reduced after a predetermined time (preferably 2 to 3 seconds) to reduce an output power, as shown in FIG. 4A. In addition, the operating frequency may also be switched to a high frequency or a low frequency after a predetermined time (preferably 2 to 3 seconds) to reduce the output power. As shown in FIG. 4B, the current signal is used as the basis for determining the operating frequency, and different frequencies are provided to obtain the feedback current signal of nebulization module 105. Controller 103 defines the highest current signal as the first operating frequency (i.e., the best operating point; “8” corresponding to the horizontal axis shown in FIG. 4B), when controller 103 adjusts the operating frequency after a specific time, because the current signal corresponding to the second operating frequency (i.e., the second best operating point; “7” corresponding to the horizontal axis shown in FIG. 4B) is higher than that corresponding to the third operating frequency (“9” corresponding to the horizontal axis shown in FIG. 4B). Controller 103 automatically selects the second operating frequency for offloading, so as to avoid heating and failure of the medicament caused by nebulization module 105 when the medicament is reduced. In general, since the nebulization effect of the third operating frequency is very weak, users may have the illusion that there is no nebulization, the third operating frequency is not used. The execution of the aforementioned offloading mechanism may be completed before aerosol medicament delivery system 10 is activated.

### EXAMPLE 2

### Offloading through Detecting a Liquid Level of a Medicament

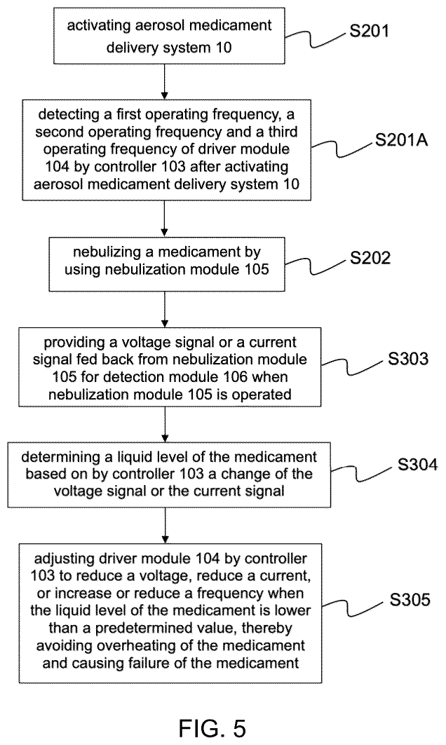

The nebulization of a conventional nebulizer is driven by a constant power. When the nebulizer filled with the medicament nebulizes after a period of time, the power driven to the nebulization module does not decrease as the amount of the medicament decreases. Thus, the heat energy of the nebulization module may cause failure of the remaining medicament. In order to reduce the waste caused by overheating of the medicament, as shown in FIG. 5, the present disclosure provides a method for offloading aerosol medicament delivery system 10 further including detection module 106, and the method further includes steps of:
S303: providing a voltage signal or a current signal fed back from nebulization module 105 for detection module 106 when nebulization module 105 is operated;
S304: determining a liquid level of the medicament by controller 103 based on a change of the voltage signal or the current signal; and
S305: adjusting driver module 104 by controller 103 to reduce a voltage, reduce a current, or increase or reduce a frequency when the liquid level of the medicament is lower than a predetermined value, thereby avoiding overheating of the medicament and causing failure of the medicament.

Preferably, S305 may further include controlling driver module 104 to drive nebulization module 105 by controller 103 to nebulize the medicament at the second operating frequency when the liquid level of the medicament is lower than the predetermined value, thereby avoiding overheating of the medicament and causing failure of the medicament.

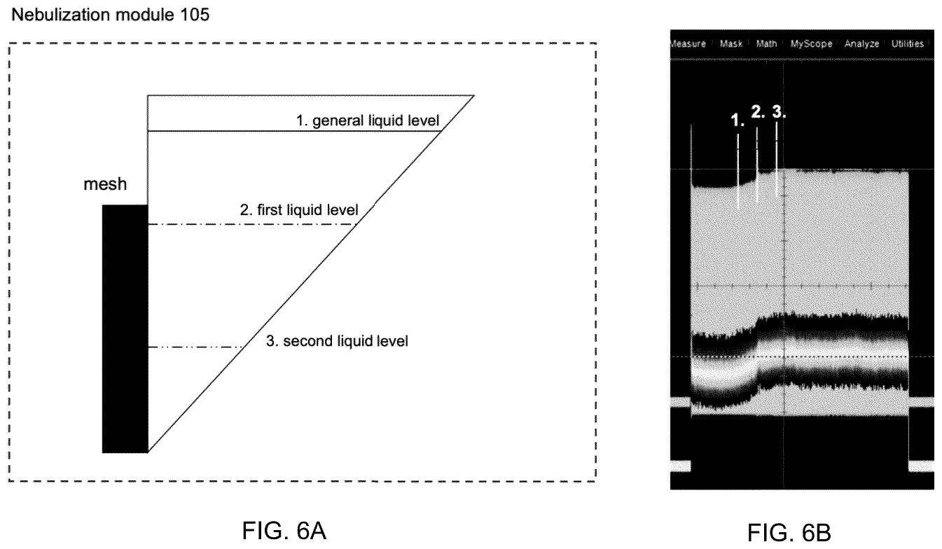

Specifically, FIG. 6A shows that a difference in the reaction forces of the three liquid levels of the medicament applied on the mesh may produce different feedback signals. The general liquid level has a greater reaction force applied on the mesh than the first liquid level, and the first liquid level has a greater reaction force on the mesh than the second liquid level. The aforementioned difference in reaction force may be reflected in the voltage and current of nebulization module 105. As shown in FIG. 6B, the feedback voltage and current signals before the medicament is almost to be exhausted show a slow-rising section. The aforementioned changes in voltage and current signals are used to confirm the liquid level of the medicament, and offloading is achieved after 1 to 2 seconds, so as to avoid failure of the medicament due to excessive energy of nebulization module 105 and overheating of the remaining medicament. The execution of the aforementioned offloading mechanism may also be completed before aerosol medicament delivery system 10 is activated.

### EXAMPLE 3

### Dosing Control of Aerosol Medicament Delivery System

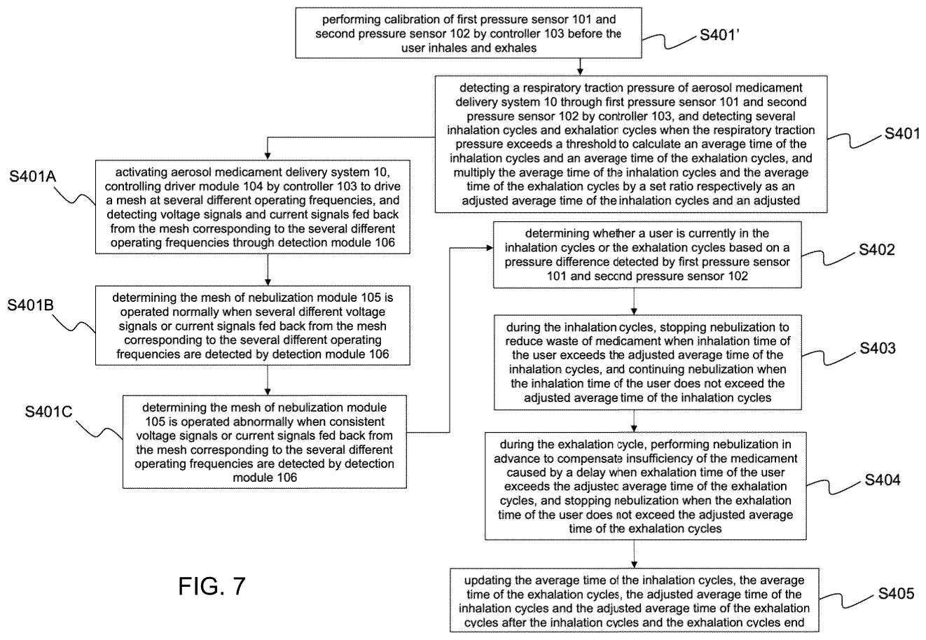

In order to improve the medicament delivery rate of aerosol drug delivery system 10, as shown in FIG. 7, one embodiment of the present disclosure provides a method for controlling nebulization of aerosol medicament delivery system 10, including steps of:
S401: detecting a respiratory traction pressure of aerosol medicament delivery system 10 through first pressure sensor 101 and second pressure sensor 102 by controller 103, and
   detecting several inhalation cycles and exhalation cycles when the respiratory traction pressure exceeds a threshold to calculate an average time of the inhalation cycles and an average time of the exhalation cycles, and multiply the average time of the inhalation cycles and the average time of the exhalation cycles by a set ratio respectively as an adjusted average time of the inhalation cycles and an adjusted average time of the exhalation cycles;
S402: determining whether a user is currently in the inhalation cycles or the exhalation cycles based on a pressure difference detected by first pressure sensor 101 and second pressure sensor 102;
S403: during the inhalation cycles, stopping nebulization to reduce waste of medicament when inhalation time of the user exceeds the adjusted average time of the inhalation cycles, and continuing nebulization when the inhalation time of the user does not exceed the adjusted average time of the inhalation cycles;
S404: during the exhalation cycle, performing nebulization in advance to compensate insufficiency of the medicament caused by a delay when exhalation time of the user exceeds the adjusted average time of the exhalation cycles, and
   stopping nebulization when the exhalation time of the user does not exceed the adjusted average time of the exhalation cycles; and
S405: updating the average time of the inhalation cycles, the average time of the exhalation cycles, the adjusted average time of the inhalation cycles and the adjusted average time of the exhalation cycles after the inhalation cycles and the exhalation cycles end.

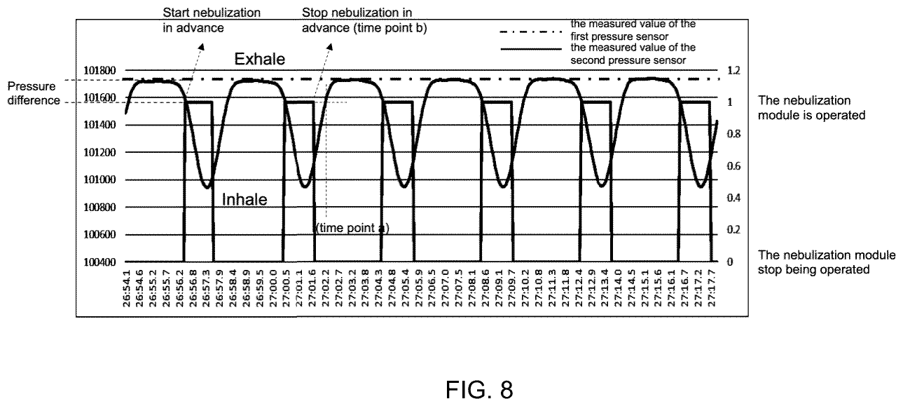

For example, as shown in FIG. 8, if the pressure difference between the measurement value of first pressure sensor 101 minus the measurement value of second pressure sensor 102 is greater than the set threshold (e.g., 60 Pa), it is determined that an inhalation cycle is about to enter, and controller 103 causes the aerosol drug delivery system 10 to start nebulization in advance. If the pressure difference between the measurement value of first pressure sensor 101 minus the measurement value of second pressure sensor 102 is lower than the set threshold (e.g., 60 Pa), it is determined that an exhalation cycle is about to enter (time point a shown in FIG. 8), and controller 103 causes the aerosol drug delivery system 10 to stop nebulization in advance (time point b shown in FIG. 8).

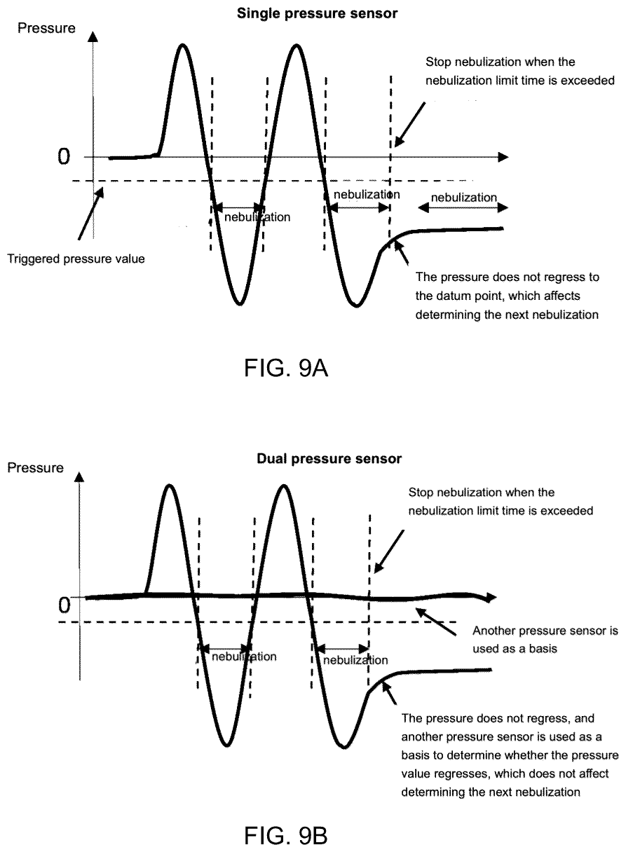

Compared with conventional pressure traction nebulizers, which uses a single pressure, differential pressure or flow sensor for nebulization, a single pressure sensor is initially triggered until the pressure reaches a threshold before subsequent actions can be activated. However, when the single pressure sensor deviates in the environment, or the pressure cannot regress normally, it will cause inaccuracy and failure (as shown in FIG. 9A). The present disclosure uses dual pressure sensors that are calibrated before use, for detecting pressure changes through the second pressure sensor is detected, when the above situation occurs in the second pressure sensor, calibrating the second pressure sensor based on the first pressure sensor, and when the first pressure sensor is out of accuracy, calibrating the first pressure sensor through the second pressure sensor regression on the contrary (as shown in FIG. 9B).

Referring back to FIG. 8, preferably before S401, the method may further include S401′: performing calibration of first pressure sensor 101 and second pressure sensor 102 by controller 103 before the user inhales and exhales.

### EXAMPLE 4

### Failure Detection of the Nebulization Module

After long-term use of the aerosol medicament delivery system, since the nebulization module has been steamed with wet heat for several times, the piezoelectric element in the nebulization module may gradually separate from the mesh, so that the reaction force of the mesh cannot be transmitted back to the detection module of the aerosol medicament delivery system, resulting in a decrease in the nebulization rate. In order to solve the aforementioned problems, the present disclosure confirms whether the failure of the nebulization module occurs through the calculation of the feedback signal of the nebulization module.

Referring to FIG. 7 again, before S401, the method may further include steps of:
S401A: activating aerosol medicament delivery system 10, controlling driver module 104 by controller 103 to drive a mesh at several different operating frequencies, and
   detecting voltage signals and current signals fed back from the mesh corresponding to the several different operating frequencies through a detection module;
S401B: determining the mesh of nebulization module 105 is operated normally when several different voltage signals or current signals fed back from the mesh corresponding to the several different operating frequencies are detected by detection module 106; and
S401C: determining the mesh of nebulization module 105 is operated abnormally when consistent voltage signals or current signals fed back from the mesh corresponding to the several different operating frequencies are detected by detection module 106.

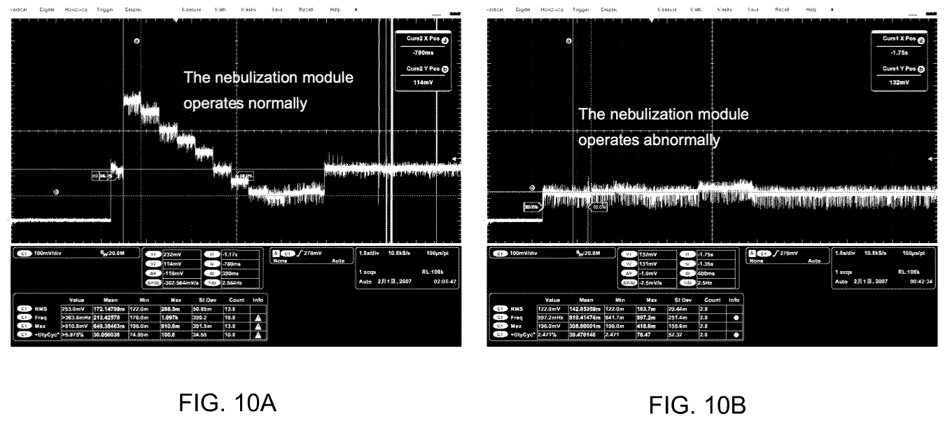

Specifically, when aerosol medicament delivery system 10 is turned on for the first time and operates at different operating frequencies, nebulization module 105 including the mesh and the piezoelectric element may feedback different voltage signals and current signals. Taking FIG. 10 as an example, which shows a current signal. As shown in FIG. 10A, a nebulization module in normal operation reflects different current signals at different operating frequencies. However, as shown in FIG. 10B, a nebulization module in abnormal operation may not reflect different current signals at different operating frequencies due to the damage of the body material. None of the above conditions will be affected by the presence or absence of the medicament.

Given the above, the advantages of the present disclosure are as follows:
(1) small size and low costs;
(2) the voltage or current can be adjusted automatically after a specific period of time, or the voltage or current can be adjusted through a change of the liquid level of the medicament fed back from the mesh to reduce the waste caused by overheating of the medicament;
(3) dual pressure sensors are used to calibrate each other to prevent one of the pressure sensors from being out of accuracy and unable to regress normally, causing abnormal nebulization; and
(4) the time to start nebulization in advance and stop nebulization in advance can be adjusted dynamically based on the user's inhalation cycles and exhalation cycles to avoid unnecessary loss of the medicament.

Although the present disclosure has been disclosed in preferred embodiments, those embodiments are not intended to limit the present disclosure. Any person skilled in the art can make various changes and modifications without departing from the spirit and scope of the present disclosure. Therefore, the protection scope of the present disclosure shall be determined by the appended claims.

1. An aerosol medicament delivery system, comprising:
a first pressure sensor configured to detect external air pressure outside the aerosol medicament delivery system;
a second pressure sensor configured to detect air pressure in an airway of the aerosol medicament delivery system;
a controller;
a driver module;
a nebulization module comprising a mesh, wherein the controller controls the driver module to drive the nebulization module for performing nebulization based on air pressure difference between the external air pressure and the air pressure in the airway;
a detection module configured to detect pressure applied by a medicament onto the mesh, convert the pressure into a voltage signal or a current signal, and transmit the voltage signal or the current signal to the controller, wherein the controller determines the liquid level of the medicament based on the voltage signal or the current signal; and
a power supply module configured to provide power for the aerosol medicament delivery system.

2. The aerosol medicament delivery system of claim 1, wherein under a condition that the air pressure difference between the external air pressure and the air pressure in the airway is greater than a first threshold, the controller drives the nebulization module to perform nebulization.

3. The aerosol medicament delivery system of claim 2, wherein the first threshold is 60 Pa.

4. The aerosol medicament delivery system of claim 1, wherein the voltage signal or the current signal is detected and fed back from the nebulization module when the nebulization module is being operated; and whether the medicament is exhausted based on a change of the voltage signal or the current signal is determined.

5. The aerosol medicament delivery system of claim 4, wherein when it is determined that the medicament is exhausted, the nebulization is stopped; and when it is determined that the medicament is not yet exhausted, the nebulization is continuing.

6. The aerosol medicament delivery system of claim 5, wherein it is determined that the medicament is exhausted when a slope of the voltage signal or the current signal corresponding to time is greater than a second threshold.

7. The aerosol medicament delivery system of claim 6, wherein the second threshold is 1.

8. A method for offloading an aerosol medicament delivery system, wherein the aerosol medicament delivery system comprises a controller and a driver module configured to drive a nebulization module for operation, and the method comprises steps of:
S201: activating the aerosol medicament delivery system;
S202: nebulizing a medicament by using the nebulization module; and
S203: adjusting the driver module by the controller to reduce an operating voltage or an operating current of the nebulization module, or increase or reduce an operating frequency of the nebulization module after the nebulization module is operated for a predetermined time.

9. The method of claim 8, after S201 and before S202, further comprising a step of:
S201A: detecting a first operating frequency, a second operating frequency and a third operating frequency of the driver module by the controller after activating the aerosol medicament delivery system;
wherein S202 further comprises controlling the driver module to drive the nebulization module by the controller to nebulize the medicament at the first operating frequency;
wherein S203 further comprises controlling the driver module to drive the nebulization module by the controller to nebulize the medicament at the second operating frequency after the nebulization module is operated for the predetermined time, thereby avoiding overheating of the medicament and causing failure of the medicament.

10. The method as claim 9, a current value corresponding to the first operating frequency is highest, a current value corresponding to the second operating frequency is second highest, and a current value corresponding to the third operating frequency is lower than the current value corresponding to the second operating frequency.

11. The method of claim 9, wherein the third operating frequency is not used for nebulization.

12. The method of claim 8, wherein the aerosol medicament delivery system further comprises a detection module, and the method further comprises steps of:
S303: providing a voltage signal or a current signal fed back from the nebulization module for the detection module when the nebulization module is operated;
S304: determining a liquid level of the medicament by the controller based on a change of the voltage signal or the current signal; and
S305: adjusting the driver module by the controller to reduce a voltage, reduce a current, or increase or reduce a frequency when the liquid level of the medicament is lower than a predetermined value, thereby avoiding overheating of the medicament and causing failure of the medicament.

13. The method of claim 12, wherein S305 further comprises: controlling the driver module to drive the nebulization module by the controller to nebulize the medicament at the second operating frequency when the liquid level of the medicament is lower than the predetermined value, thereby avoiding overheating of the medicament and causing failure of the medicament.

14. The method of claim 13, wherein a current value corresponding to the first operating frequency is highest, a current value corresponding to the second operating frequency is second highest, and a current value corresponding to the third operating frequency is lower than the current value corresponding to the second operating frequency.

15. The method of claim 13, wherein the third operating frequency is not used for nebulization.

16. A method for controlling nebulization of an aerosol medicament delivery system, comprising steps of:
S401: detecting a respiratory traction pressure of the aerosol medicament delivery system through a first pressure sensor and a second pressure sensor by a controller, and
detecting several inhalation cycles and exhalation cycles when the respiratory traction pressure exceeds a threshold to calculate an average time of the inhalation cycles and an average time of the exhalation cycles, and multiply the average time of the inhalation cycles and the average time of the exhalation cycles by a set ratio respectively as an adjusted average time of the inhalation cycles and an adjusted average time of the exhalation cycles;
S402: determining whether a user is currently in the inhalation cycles or the exhalation cycles based on a pressure difference detected by the first pressure sensor and the second pressure sensor;
S403: during the inhalation cycles, stopping nebulization to reduce waste of medicament when inhalation time of the user exceeds the adjusted average time of the inhalation cycles, and
continuing nebulization when the inhalation time of the user does not exceed the adjusted average time of the inhalation cycles;
S404: during the exhalation cycle, performing nebulization in advance to compensate insufficiency of the medicament caused by a delay when exhalation time of the user exceeds the adjusted average time of the exhalation cycles, and
stopping nebulization when the exhalation time of the user does not exceed the adjusted average time of the exhalation cycles; and
S405: updating the average time of the inhalation cycles, the average time of the exhalation cycles, the adjusted average time of the inhalation cycles and the adjusted average time of the exhalation cycles after the inhalation cycles and the exhalation cycles end.

17. The method of claim 16, before S401, further comprising steps of:
S401A: activating the aerosol medicament delivery system,
controlling a driver module by a controller to drive a mesh at several different operating frequencies, and
detecting voltage signals and current signals fed back from the mesh corresponding to the several different operating frequencies through a detection module;
S401B: determining the mesh of the nebulization module is operated normally when several different voltage signals or current signals fed back from the mesh corresponding to the several different operating frequencies are detected by the detection module; and
S401C: determining the mesh of the nebulization module is operated abnormally when consistent voltage signals or current signals fed back from the mesh corresponding to the several different operating frequencies are detected by the detection module.

18. The method of claim 16, wherein the set ratio is 80% of the average time of the inhalation cycles.

19. The method of claim 16, before S401, further comprising a step of:
S401′: performing calibration of the first pressure sensor and the second pressure sensor by the controller before the user inhales and exhales.

20. The method of claim 16, wherein the voltage signals and current signals fed back from the mesh corresponding to the several different operating frequencies are not affected by the dosage of the medicament.
